(19) United States
(12) Patent Application Publication
(10) Pub. No.: US 2008/0234648 A1
(43) Pub. Date: Sep. 25, 2008

(54) Disposable diaper with adornment feature

(76) Inventor: Sue L. Dunham, Marlin, TX (US)

Correspondence Address: ARNOLD & KNOBLOCH, L.L.P., 2401 FOUNTAIN VIEW DRIVE, SUITE 630, HOUSTON, TX, 77057, US

(21) Appl. No.: 11/726,959

(22) Filed: Mar. 21, 2007

(51) Int. Cl. A61F 13/15

(52) U.S. Cl. 604/385.13

(57) ABSTRACT

A disposable diaper having an adornment feature on a rear panel thereof, rendering the diaper more aesthetically pleasing than a conventional disposable diaper. In one embodiment, the disposable diaper includes a real panel having an adornment of ruffled material carried thereon. The adornment feature conceals the outer surface of said real panel. In a preferred embodiment, the adornment feature does not substantially increase the folded volume of the diaper.

### FIELD OF THE INVENTION

The present invention relates generally to the field of infant/toddler apparel, and more particularly relates to disposable diapers worn by infants/toddlers.

### BACKGROUND OF THE INVENTION

Disposable diapers are commercially-available from innumerable sources, and are available in an almost endless variety of configurations, incorporating various features and being composed of a range of combinations of materials. Those of ordinary skill in the art, a class which for the purposes of the present disclosure shall be considered to include a majority of parents of young children (infants and toddlers) who wear disposable diapers, will be very familiar with disposable diapers of various types.

Any parent in the relevant class will appreciate that young children (infants and toddlers) wear diapers to absorb and/or contain a child's urinary and defacatory output, until such time as the child is trained to control his or her urinary and defactory functions to the point that he or she is capable of utilizing a conventional commode without soiling his or herself. (This is typically referred to as “potty training”).

The age at which a child achieves a consistent level of “potty training” so that the need for diapers is eliminated can vary. Some children achieve consistent “potty training” at a relatively early age, while others may take longer.

During the time period in which a child is expected to wear diapers, by far the most prevalent approach involves the use of disposable diapers. The era of cloth diapers, requiring aggressive laundering and posing challenges relating to their containment prior to laundering, is seemingly coming to an end. The use of non-disposable diapers involves ensuring that a supply of diapers is available at all times, and further that a means for containing soiled diapers prior to laundering is available.

The difficulties associated with the use of non-disposable (e.g., cloth) diapers eventually led to the development and widespread popularity of disposable diapers, commercially available from a number of sources and very familiar to those of ordinary skill. Many parents believe that the disposable diaper is one of the great inventions of the 20th century—a revolutionary product which has brought huge benefits to society in terms of health, hygiene and convenience. Parents, babies and toddlers around the world have been enjoying the freedom and comfort of disposable diapers for over fifty years. Disposable diapers have overtaken the cloth diaper market by an enormous margin, and have put many cloth diaper services out of business due to their convenience and relatively small bulk as worn on the baby. Approximately 18 billion units of disposable diapers were sold in the United States in 2004.

Although countless varieties of disposable diapers are known and commercially available, there are some basic features that are for the most part common to virtually any example of a disposable diaper. Modern disposable diapers are generally made of a cloth-like, waterproof exterior, a moisture-wicking inside layer, and an absorbent inner core (in modern diapers usually a dried, superabsorbant hydrogel).

FIG. 1 is a partially cut-away view of the interior surface 12 of a typical prior art disposable diaper 10 shown after being unfolded. (Those of ordinary skill in the art will be aware that disposable diapers are typically packaged for retail sale in a folded condition in order to reduce the volume of the product during shipment and retail display.) As shown in FIG. 1, diaper 10 includes comprises a contoured form including an inner, absorptive layer, designated with reference numeral 12 in FIG. 1, and an outer layer 14 often selected to be impermeable to moisture and the like. Sandwiched between inner layer 12 and outer layer 14 is an absorptive inner core 16, such as a hydrogel.

Diaper 10 comprises essentially three regions, namely a first region 18 corresponding to the rear or seat panel of the diaper 10, a second region 20 corresponding to the front panel of diaper 10, and a central portion 22 corresponding to a bottom portion of diaper 10. As shown in FIG. 1, central portion 22 is contoured and may have thigh-surrounding elastic members 24. Reference numeral 26 denotes the rear waist edge of diaper 10, and reference numeral 28 denotes the front waist edge of 11 diaper 10. Diaper 12 is provided with a pair of tape fasteners 30 proximal the rear waist edge 26.

FIG. 2 is an isometric view of diaper 10 depicting how it is worn. As can be seen in FIG. 2, diapering a child entails positioning seat panel 18 behind the child' buttocks and folding diaper 10 to bring the front panel 20 to the front of the child. Diaper 10 is secured to the child by affixing tape fasteners 30 to the front panel 20.

FIG. 3 is a view of the exterior surface 12 of prior art diaper 10. In the prior art, the material comprising the exterior surface 12 may have colors, patterns, or child-appropriate illustrations imprinted thereon, most commonly on the respective upper portions of seat panel 18 and front panel 20, to improve the aesthetic appeal of the diaper to children and parents alike.

Various methods of manufacture of a diaper such as diaper 10 described herein are known to those in the industry. U.S. Pat. No. 7,132,031 to Ohiro et al, entitled “Process for Making a Disposable Diaper” is but one example. The Ohiro et al. '031 patent is hereby incorporated by reference herein in its entirety.

Those of ordinary familiarity with disposable diapers having the need to utilize such diapers for their children, will recognize that it is not uncommon for the disposable diaper to serve as the only clothing covering the child from the waist down. That is, parents and others many times do not go to the effort to add additional clothing such as pants, dresses, or the like overtop the diaper. Depending on the circumstances, it may be undesirable or unnecessary to provide additional clothing over the diaper, particularly for example when it is necessary to change the diaper frequently, or when the child is in relatively informal settings.

### SUMMARY OF THE INVENTION

In recognition of the fact that disposable diapers oftentimes serve as the outermost layer of a child's clothing from the waist down, the present invention is directed to a disposable diaper having additional adornment on at least the rear panel thereof, thereby enhancing the aesthetic appeal of the diaper. A significant advantage of the invention is that it is less obvious to casual observers that a child may be wearing only a diaper, without additional clothing, from the waist down.

In one embodiment, a disposable diaper is provided with a rear panel having a ruffled adornment feature thereon. The adornment feature can be made of material the same or similar to the material normally used as the outer layer of a disposable diaper, and can be of attractive colors more pleasing to the eye than the bare white rear panel of many disposable diapers, or the simply colored or illustrated rear panel of other disposable diapers.

In accordance with one beneficial aspect of the invention, the adornment increases the aesthetic appeal of a disposable diaper, without a substantial increase in the size of the diaper when folded for packaging, shipping, and retail display.

In accordance with another beneficial aspect of the invention, providing the additional adornment can be accomplished as part of conventional diaper manufacturing processes, and can be accomplished with only marginal, if any, increase in the cost of manufacture.

### BRIEF DESCRIPTION OF THE DRAWINGS

The foregoing and other features and aspects of the present invention will be best appreciated by reference to a detailed description of the specific embodiments of the invention, when read in conjunction with the accompanying drawings, wherein:

FIG. 1 is a view of the inner surface of a prior art disposable diaper;

FIG. 2 is an isometric view of the prior art diaper of Figure shown as it would be worn by a child;

FIG. 3 is a view of the outer surface of the prior art disposable diaper;

FIG. 4 is a view of the outer surface of a diaper in accordance with one embodiment of the invention;

FIG. 5 is a rear isometric view of the diaper from FIG. 4;

FIG. 6 is a side view of the diaper from FIG. 4; and

FIG. 7 is a top view showing the manner of creating an adornment in accordance with one embodiment of the invention.

### DETAILED DESCRIPTION OF A SPECIFIC EMBODIMENT OF THE INVENTION

In the disclosure that follows, in the interest of clarity, not all features of actual implementations are described. It will of course be appreciated that in the development of any such actual implementation, as in any such project, numerous engineering and technical decisions must be made to achieve the developers' specific goals and subgoals (e.g., compliance with manufacturing constraints), which will vary from one implementation to another. Moreover, attention will necessarily be paid to proper practices for the environment in question. It will be appreciated that such a development effort might be complex and time-consuming, but would nevertheless be a routine undertaking for those of ordinary skill in the relevant fields.

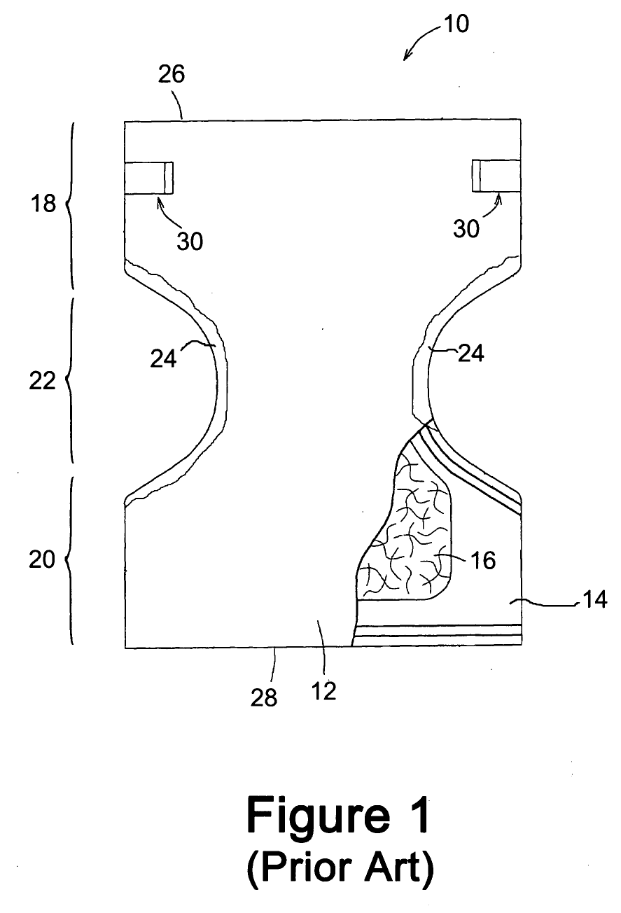

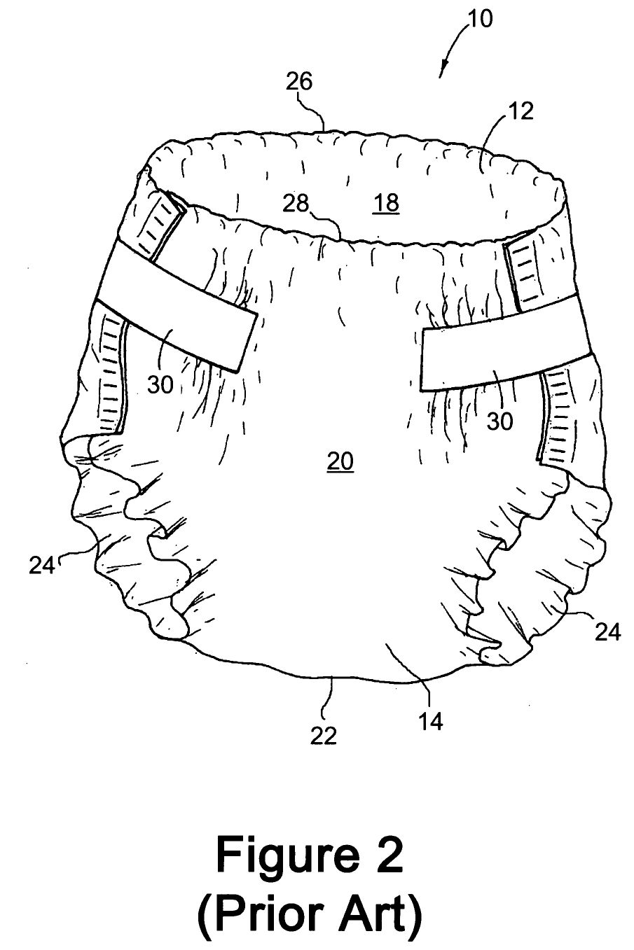

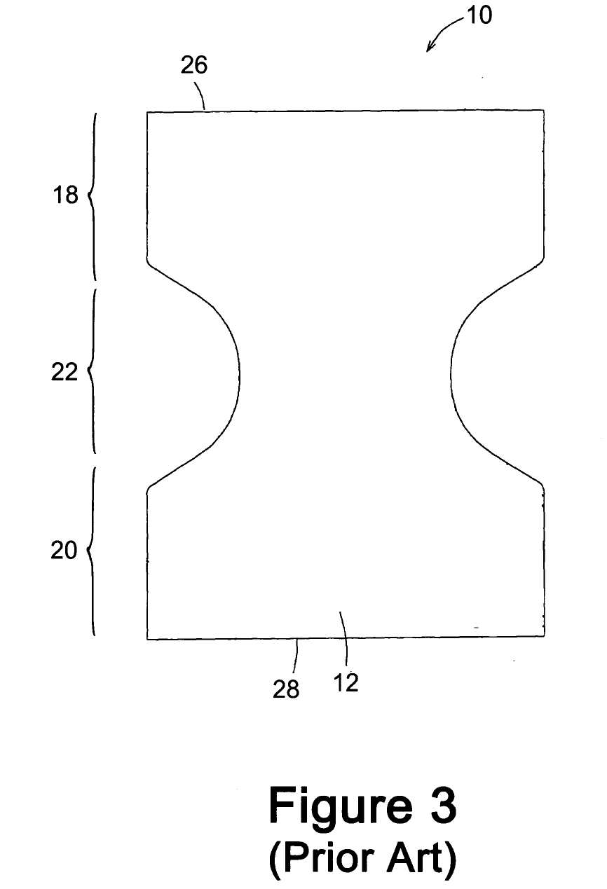

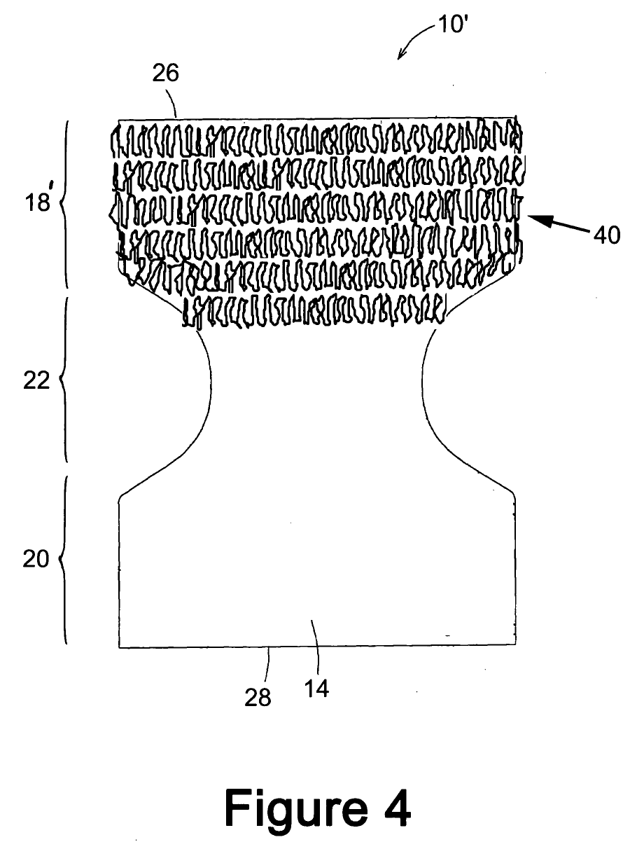

Referring to FIG. 4, there is shown a view of the outer surface 14 of a disposable diaper 10′ in accordance with a presently preferred embodiment of the invention. It is to be understood that in FIG. 4 and the Figures that follow, elements of diaper 10′ that are essentially identical to those in the prior art diaper of FIGS. 1 through 3 will retain identical reference numerals.

As shown in FIG. 4, diaper 10′ comprises essentially the same front panel 20 and bottom portion 22 previously described. However, bottom or seat panel 18 is distinguished in the present invention by virtue of it having an adornment feature 40 applied thereon. Specifically, in the presently disclosed embodiment, adornment 40 comprises an area of ruffled or gathered material as might be found on more formal types of clothing. In accordance with a principal aspect of the invention, the adornment 40 serves to conceal the outer surface 14 of diaper 10′, rendering diaper 10′ less readily identifiable as a disposable diaper and more likely to a casual observer to be an article of apparel other than a disposable diaper.

In the presently disclosed embodiment, ruffled adornment 40 is composed of Pellon®, a well-known variety of non-woven fabric (sometimes referred to as “interfacing”). Pellon® brand products are distributed in the United States and Canada through PCP Group LLC (Pellon® Consumer Products), in Tucker, Ga. Pellon® brand products are readily available on a retail basis at fabric outlets and the like. Pellon® brand products are most commonly supplied in white, but the inventors have shown that the material can be quite readily dyed to any desired color using conventional off-the-shelf dyes, as would be a matter of routine practice to those of ordinary skill.

In accordance with one aspect of the invention, the outer layer 14 of conventional disposable diapers is often composed of a material quite similar to, if not identical, to Pellon®. Hence, the present invention can be practiced with minimal changes to conventional manufacturing processes and using the same or similar materials as in prior art disposable diapers. Moreover, with the adornment 40 composed of Pellon®, the adornment 40 is easily compressed, such that a diaper in accordance with the present invention does not occupy substantially more volume than a prior art diaper for the purposes of packaging, shipping, and retail display. This is a significant advantage, inasmuch as the compactness of disposable diapers as a retail commodity is of particular concern, as is explained in greater detail in Malcolm Gladwell, “Annals of Technology: ‘Smaller: The Disposable Diaper and the Meaning of Progress;’” The New Yorker, Nov. 26, 2001, pp. 74-79 (“Gladwell”). Gladwell is hereby incorporated by reference herein in its entirety.

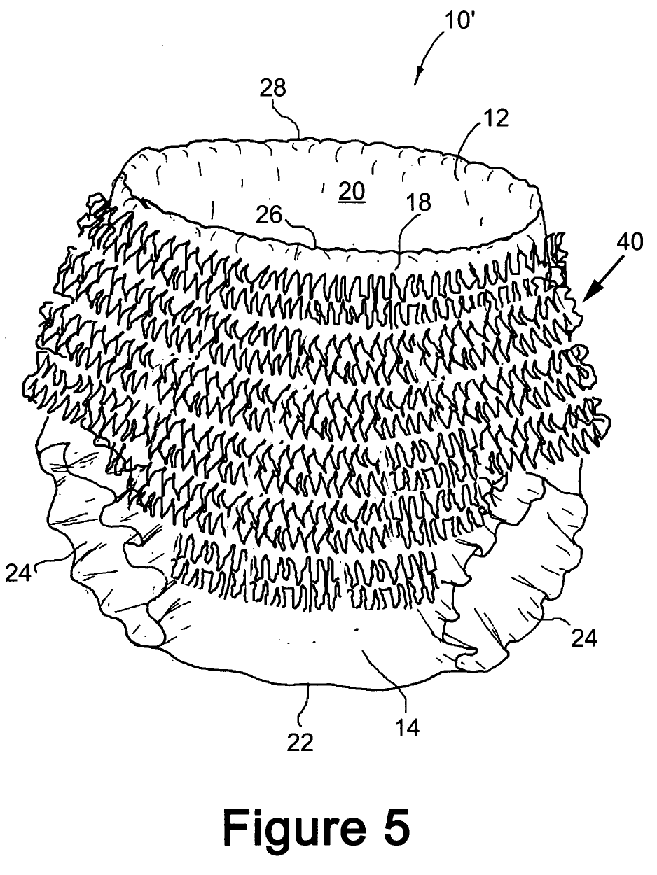

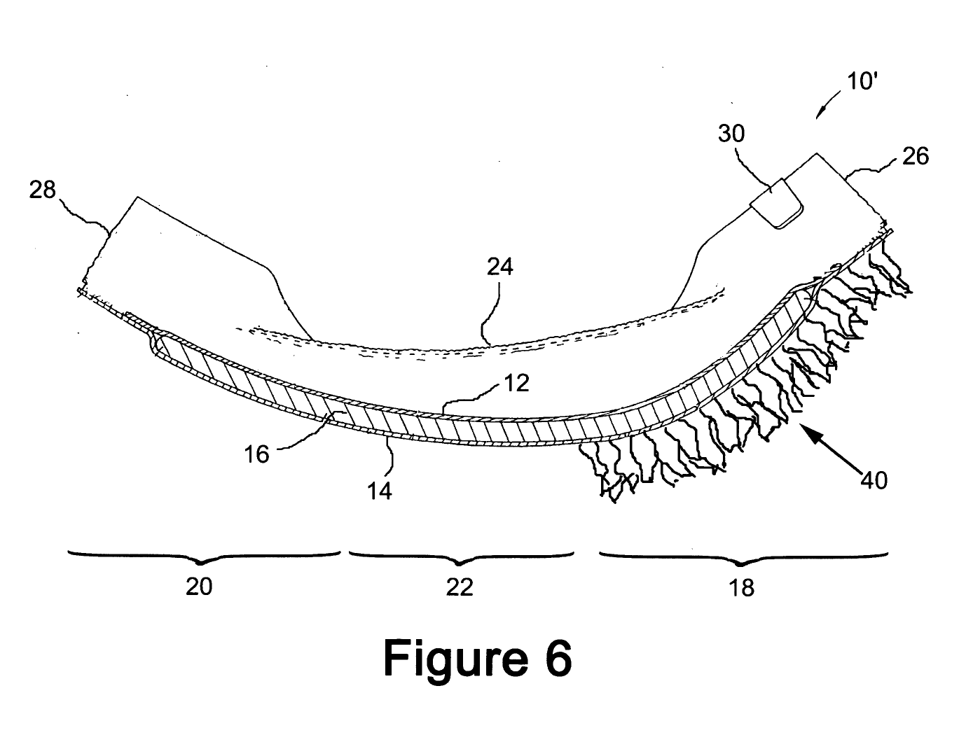

FIG. 5 is an isometric rear view of diaper 10′ in accordance with the presently disclosed embodiment of the invention. FIG. 6 is a side view of diaper 10′ in accordance with the presently disclosed embodiment of the invention.

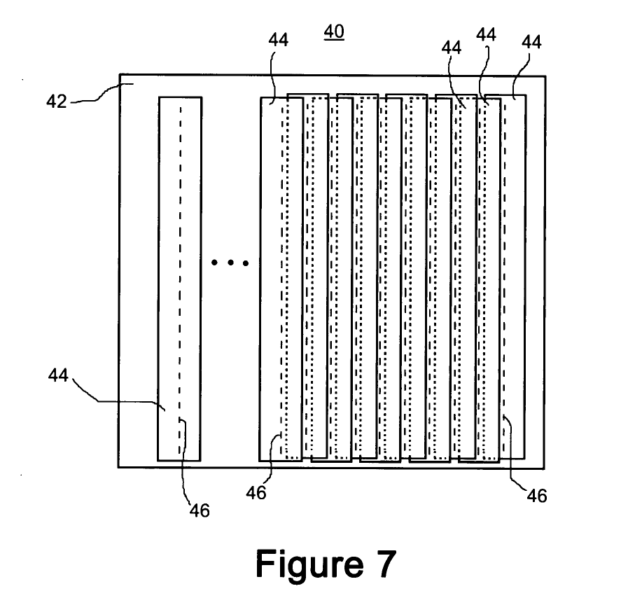

Referring to FIG. 7, there is depicted one method for creating adornment 40 in accordance with the invention. As shown in FIG. 7, adornment 40 comprises a base layer 42 having a plurality of elongate strips 44 affixed thereto. An exemplary strip 44 is shown in isolation in FIG. 7. As can be seen, strips 44 are affixed to base layer 42 by stitching 46 extending along the length of each strip 44. Notably, stitching 44 is performed using an elastic thread such that once strips 44 are in place, the stitching causes both the strips 44 and base layer 42 to contract, forming the ruffled appearance as shown in FIGS. 4 through 6.

In the presently preferred embodiment, strips 44 are on the order of 1½ inches wide, while each stitching row 46 is spaced apart approximately ¾ inch or less from each adjacent stitching row 46. Thus, as shown in FIG. 7, adjacent strips 44 overlap one another, to ensure that a ruffled effect is achieved upon contraction of the elastic stitching 46.

In one embodiment, base layer 42 is the material serving as outer layer 14 of diaper 10′, i.e., the ruffled adornment 40 is formed directly upon the outer surface of diaper 10′. Alternatively, ruffled adornment 40 may be formed on a separate base layer 42 and subsequently applied to the outer surface 14 of diaper 10′ by appropriate means, which may include, by way of example but not limitation, fabric adhesives, stitching, or other glues, as would be apparent to those of ordinary skill.

From the foregoing detailed description, it should be apparent that a disposable diaper having an adornment feature thereon has been disclosed. Although a specific embodiment of the invention has been described herein, it is to be understood that this has been done solely for the purposes of illustrating various features and aspects of the invention, and is not intended to be limiting with respect to the scope of the invention, as defined in the claims. It is contemplated and to be understood that various substitutions, alterations, and/or modifications, including such implementation variants and options as may have been specifically noted or suggested herein, may be made to the disclosed embodiment of the invention without departing from the spirit or scope of the invention.

What is claimed is:

1. A disposable diaper, comprising:
a contoured form having an inner surface and an outer surface, said contoured form adapted to be arranged around the waist of a wearer such that a rear panel of said outer surface is exposed at the rear of the wearer;
an adornment feature, disposed on said rear panel, said adornment feature adapted to conceal said rear panel of said outer surface.

2. A disposable diaper in accordance with claim 1, wherein said adornment feature comprises an area of ruffled material.

3. A disposable diaper in accordance with claim 2, wherein said ruffled material comprises a gathered fabric.

4. A disposable diaper in accordance with claim 3, wherein said gathered fabric comprises a non-woven material.

5. A disposable diaper in accordance with claim 3, wherein gathered fabric comprises a plurality of elongate strips of material affixed to a base layer.

6. A disposable diaper in accordance with claim 5, wherein said elongate strips are affixed to said base layer in a manner causing said elongate strips to gather into a ruffled configuration.

7. A disposable diaper in accordance with claim 6, wherein said elongate strips are affixed to said base layer by means of stitching.

8. A disposable diaper in accordance with claim 7, wherein said stitching includes an elastic thread.

9. A disposable diaper in accordance with claim 5, wherein said base layer comprises said outer surface of said rear panel.

10. A disposable diaper in accordance with claim 6, wherein said base layer comprises a layer of material separate from said outer surface of said rear panel.

11. A disposable diaper in accordance with any of claims 1 through 10, wherein said adornment feature is comprised of Pellon® non-woven fabric.
